(19) United States
(12) Patent Application Publication
(10) Pub. No.: US 2021/0406586 A1
(43) Pub. Date: Dec. 30, 2021

(54) IMAGE CLASSIFICATION METHOD AND APPARATUS, AND STYLE TRANSFER MODEL TRAINING METHOD AND APPARATUS

(71) Applicant: Beijing Baidu Netcom Science and Technology Co., LTD, Beijing (CN)

(72) Inventors: Dalu YANG, Beijing (CN); Yehui YANG, Beijing (CN); Lei WANG, Beijing (CN); Yanwu XU, Beijing (CN)

(21) Appl. No.: 17/139,069

(22) Filed: Dec. 31, 2020

(30) Foreign Application Priority Data
Jun. 24, 2020 (CN) 202010591392.9

(51) Int. Cl. G06K 9/62; G06K 9/46; G06N 3/08; G06N 3/04

(52) U.S. Cl. CPC G06K 9/627; G06K 9/6256; G06K 9/4652; G06K 2209/05; G06K 9/4647; G06N 3/088; G06N 3/0454; G06K 9/6262

(57) ABSTRACT

An image classification method and apparatus, and a style transfer model training method and apparatus are provided, which are relate to the field of deep learning, cloud computing and computer vision in artificial intelligence. The image classification method comprises: inputting an image of a first style into a style transfer model, to obtain an image of a second style corresponding to the image of the first style; and inputting the image of the second style into an image classification model, to obtain a classification result of the image of the second style, wherein the style transfer model is obtained through training on the basis of a sample image of the first style and a sample image of the second style; and the image classification model is obtained through training on the basis of the sample image of the second style.

### CROSS-REFERENCE TO RELATED APPLICATION

This application claims priority to Chinese Patent Application No. 202010591392.9, filed on Jun. 24, 2020, which is hereby incorporated by reference in its entirety.

### TECHNICAL FIELD

The application relates to the field of artificial intelligence, more particularly, to the field of deep learning, cloud computing and computer vision in artificial intelligence.

### BACKGROUND

In the application of a deep learning model, sample data in a training set employed in the training process of the model is sometimes inconsistent in style with data processed in the actual use of the model.

### SUMMARY

An image classification method and apparatus, and a style transfer model training method and apparatus are provided according to embodiments of the application.

In first aspect, an image classification method is provided according to an embodiment of the application, which includes:

inputting an image of a first style into a style transfer model, to obtain an image of a second style corresponding to the image of the first style; and

inputting the image of the second style into an image classification model, to obtain a classification result of the image of the second style, wherein

the style transfer model is obtained through training on the basis of a sample image of the first style and a sample image of the second style; and the image classification model is obtained through training on the basis of the sample image of the second style.

In a second aspect of the application, a style transfer model training method is provided according to an embodiment of the application, which includes:

setting a first residual generation network, a second residual generation network, an image feature extractor, a first discriminator and a second discriminator, wherein the first residual generation network is configured for generating a residual of a transfer from an image of a first style to an image of a second style; the second residual generation network is configured for generating a residual of a transfer from the image of the second style to the image of the first style; the image feature extractor is configured for extracting feature information of the image of the first style or the image of the second style; the first discriminator is configured for determining whether an image is the image of the first style; and the second discriminator is configured for determining whether the image is the image of the second style;

inputting training samples, and calculating a loss function by using output results of the first residual generation network, the second residual generation network, the first discriminator, and the second discriminator; and

adjusting a parameter in at least one of the first residual generation network, the second residual generation network, the first discriminator and the second discriminator, to cause a value of the loss function to approach an optimal value.

In a third aspect, an image classification apparatus is provided according to an embodiment of the application, which includes:

a first input module configured for inputting an image of a first style into a style transfer model, to obtain an image of a second style corresponding to the image of the first style; and

a second input module configured for inputting the image of the second style into an image classification model, to obtain a classification result of the image of the second style, wherein

the style transfer model is obtained through training on the basis of a sample image of the first style and a sample image of the second style; and the image classification model is obtained through training on the basis of the sample image of the second style.

In a fourth aspect, a style transfer model training apparatus is provided according to an embodiment of the application, which includes:

a setup module configured for setting a first residual generation network, a second residual generation network, an image feature extractor, a first discriminator and a second discriminator, wherein the first residual generation network is configured for generating a residual of a transfer from an image of a first style to an image of a second style; the second residual generation network is configured for generating a residual of a transfer from the image of the second style to the image of the first style; the image feature extractor is configured for extracting feature information of the image of the first style or the image of the second style; the first discriminator is configured for determining whether an image is the image of the first style; and the second discriminator is configured for determining whether the image is the image of the second style;

a calculation module configured for inputting training samples, and calculating a loss function by using output results of the first residual generation network, the second residual generation network, the first discriminator, and the second discriminator; and

an optimization module configured for adjusting a parameter in at least one of the first residual generation network, the second residual generation network, the first discriminator, and the second discriminator, to cause a value of the loss function to approach an optimal value.

In a fifth aspect, an electronic device is provided according to an embodiment of the application, which includes:

at least one processor; and

a memory communicatively connected with the at least one processor, wherein

the memory stores instructions executable by the at least one processor, and the instructions, when executed by the at least one processor, enable the at least one processor to perform the method according to any one of the above embodiments.

In a sixth aspect, a non-transitory computer-readable storage medium storing computer instructions is provided according to an embodiment of the application, wherein the computer instructions cause a computer to perform the method according to any one of the above embodiments.

According to the embodiments of the application, the image of the first style is subjected to a style transfer implemented by the style transfer model, and the obtained image of the second style is input to the image classification model, wherein the image classification model is obtained through training on the basis of the sample image of the second style.

It is to be understood that the description in this section is not intended to identify key or critical features of the embodiments of the application, nor is it intended to limit the scope of the application. Other features of the application will become readily apparent from the following description.

### BRIEF DESCRIPTION OF THE DRAWINGS

The drawings are used for providing a better understanding of the technical solution and are not to be construed as limiting the application, wherein

FIG. 1 is a schematic flowchart of an implementation of an image classification method according to an embodiment of the application;

FIG. 2 is a schematic diagram of a style transfer network architecture according to an embodiment of the application;

FIG. 3 is a schematic flowchart of an implementation of a style transfer model training method according to an embodiment of the application;

FIG. 4 is a schematic diagram of calculation of a loss function of a style transfer model according to an embodiment of the application;

FIG. 5 is a schematic flowchart of an implementation process for calculating a cyclic loss function in a style transfer model training method according to an embodiment of the application;

FIG. 6 is a schematic flowchart of an implementation process for calculating an adversarial loss function in a style transfer model training method according to an embodiment of the application;

FIG. 7 is a schematic diagram showing a structure of an image classification apparatus according to an embodiment of the application;

FIG. 8 is a schematic diagram showing a structure of a style transfer model training apparatus according to an embodiment of the application;

FIG. 9 is a schematic diagram showing a structure of another style transfer model training apparatus according to an embodiment of the application; and

FIG. 10 is a block diagram of an electronic device for implementing embodiments of the application.

### DETAILED DESCRIPTION

Reference will now be made in detail to the accompanying drawings to illustrate exemplary embodiments of the application, wherein the various details of the embodiments of the application are included to facilitate understanding and are to be considered as exemplary only. Accordingly, a person of ordinary skill in the art appreciates that various changes and modifications can be made to the embodiments described herein without departing from the scope and spirit of the application. Also, descriptions of well-known functions and structures are omitted from the following description for clarity and conciseness.

An image classification method is provided according to an embodiment of the application, which can solve the problem of low accuracy of an image classification model caused by a difference in image style between a training set and an actual scenario. The embodiments of the application design a style transfer model to transform a style of an image in the actual scenario into a style of an image in the training set; and by inputting the image after the style transfer (i.e., style transformation) into the image classification model, a classification result with high accuracy can be obtained.

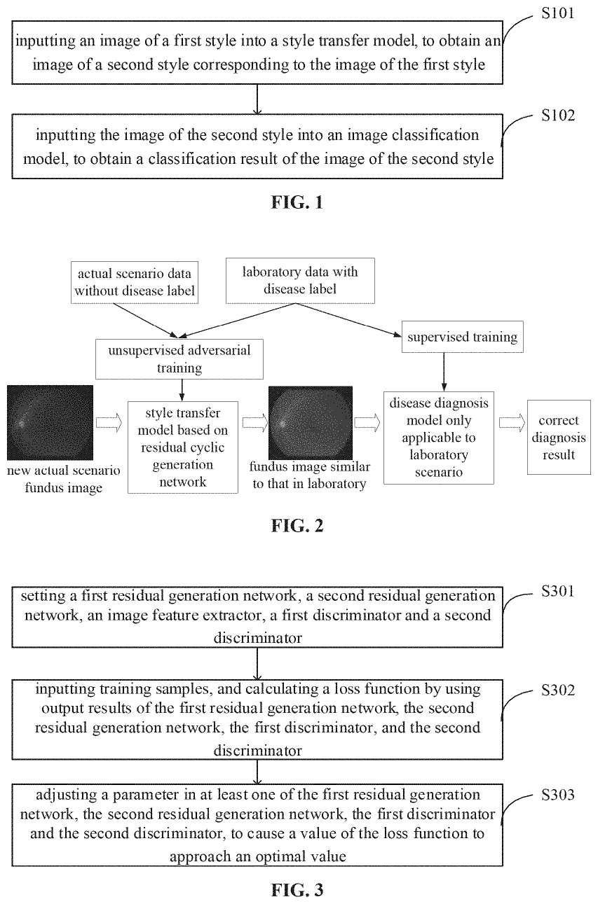

FIG. 1 is a schematic flowchart of an implementation of an image classification method according to an embodiment of the application, the image classification method including:

S101, inputting an image of a first style into a style transfer model, to obtain an image of a second style corresponding to the image of the first style; and

S102, inputting the image of the second style into an image classification model, to obtain a classification result of the image of the second style, wherein

the style transfer model is obtained through training on the basis of a sample image of the first style and a sample image of the second style; and the image classification model is obtained through training on the basis of the sample image of the second style.

The style of the image may be related with the type of a camera used for capturing the image, for example, the image of the first style includes an image captured by a camera of a first type, and the image of the second style includes an image captured by a camera of a second type.

The image of the second style corresponding to the image of the first style includes: an image having the same image content as the image of the first style and conforming to a shooting style of the camera of the second type.

That is to say, for the same target, the image captured by the camera of the first type is the image of the first style, and the image captured by the camera of the second type is the image of the second style; these two images have the same content, so they correspond to each other. The style transfer model can be used for performing a style transfer on the image of the first style and transforming the image of the first style into the corresponding image of the second style. Because the image classification model is obtained through training on the basis of the sample image of the second style, the classification accuracy in the actual scenario can basically reach the classification accuracy in the laboratory, so that the image classification accuracy is improved.

Taking the application of the image classification model to the diagnosis of a fundus image as an example, in some embodiments, the image classification model is a disease diagnosis model. FIG. 2 is a schematic diagram of a style transfer network architecture according to an embodiment of the application. In FIG. 2, the first style is specifically a style of the fundus image in the actual scenario, and the second style is specifically a style of the fundus image in the laboratory.

FIG. 2 mainly shows the following three steps:

firstly, the style transfer model may be obtained through unsupervised adversarial training on the basis of the actual scenario data without a disease label and the laboratory data with the disease label;

secondly, the disease diagnosis model adapted to use in the laboratory may be obtained through common supervised training on the basis of the laboratory data with the disease label; alternatively, the disease diagnosis model is a convolutional neural network; and

thirdly, the fundus image in the actual scenario is input into the style transfer model in the first step, to obtain an fundus image similar to that in the laboratory; and then the transferred fundus image is input into the disease diagnosis model in the second step, to obtain a high-accuracy diagnosis result (the diagnosis result may be considered as a classification result).

In some embodiments, the style transfer model includes a residual generation network including a convolution layer and a pooling layer; and the image classification model includes a convolutional neural network.

A style transfer model training method is also provided according to an embodiment of the application. FIG. 3 is a schematic flowchart of an implementation of a style transfer model training method according to an embodiment of the application, the style transfer model training method including:

S301, setting a first residual generation network, a second residual generation network, an image feature extractor, a first discriminator and a second discriminator; wherein

the first residual generation network is configured for generating a residual of a transfer from an image of a first style to an image of a second style;

the second residual generation network is configured for generating a residual of a transfer from the image of the second style to the image of the first style;

the image feature extractor is configured for extracting feature information of the image of the first style or the image of the second style;

the first discriminator is configured for determining whether an image is the image of the first style; and

the second discriminator is configured for determining whether the image is the image of the second style;

S302, inputting training samples, and calculating a loss function by using output results of the first residual generation network, the second residual generation network, the first discriminator, and the second discriminator; and

S303, adjusting a parameter in at least one of the first residual generation network, the second residual generation network, the first discriminator and the second discriminator, to cause a value of the loss function to approach an optimal value.

In some embodiments, a first style transfer model is used for performing a style transfer on the image of the first style to obtain the corresponding image of the second style; and the first style transfer model includes the first residual generation network. A second style transfer model is used for performing a style transfer on the image of the second style to obtain the corresponding image of the first style; and the second style transfer model includes the second residual generation network.

In some embodiments, S302 and S303 described above may be repeated. For example, a batch of training samples are input and the loss function is calculated, and parameters in the networks are adjusted to cause the loss function to approach an optimal value; a batch of training samples are input again and a loss function is calculated, and the parameters are adjusted again cause the loss function to approach an optimal value; and the training of the style transfer model proceeds until the value of the loss function reaches an desired range.

In some embodiments, the loss function includes a cyclic loss function and an adversarial loss function.

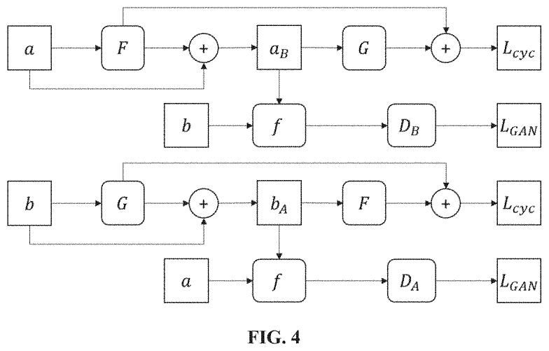

FIG. 4 is a schematic diagram of calculation of a loss function of a style transfer model according to an embodiment of the application.

FIG. 4 is explained as follows:

a: an image from a camera of type A, corresponding to the image of the first style.

b: an image from a camera of type B, corresponding to the image of the second style.

F: a first residual generation network, composed of a convolution layer and a pooling layer, for generating a residual of a transfer from type A to type B.

G: a second residual generation network, composed of a convolution layer and a pooling layer, for generating a residual of a transfer from type B to type A.

a_{B}: image a transferred to a style of type B.

b_{A}: image b transferred to a style of type A.

f: a feature extractor for a specific type.

D_{A}: a discriminator for determining whether an image comes from type A, corresponding to the first discriminator.

D_{B}: a discriminator for determining whether an image comes from type B, corresponding to the second discriminator.

L_{cyc}: a cyclic loss, which is used to direct the image having experienced two rounds (F and G) of transfer to be as similar as the original image as possible. Two cyclic losses are shown in FIG. 4, namely, a first cyclic loss and a second cyclic loss.

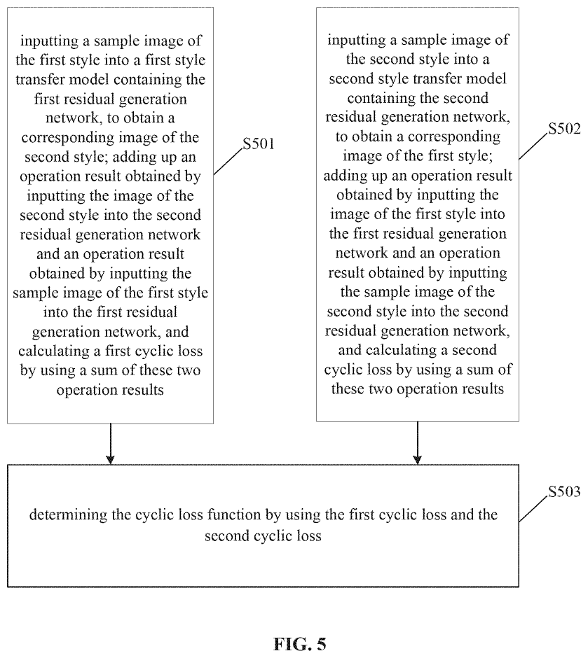

Referring to FIG. 4, in some embodiments, a process for calculating the cyclic loss function is shown in FIG. 5, including:

S501, inputting a sample image of the first style into a first style transfer model containing the first residual generation network, to obtain a corresponding image of the second style; adding up an operation result obtained by inputting the image of the second style into the second residual generation network and an operation result obtained by inputting the sample image of the first style into the first residual generation network, and calculating a first cyclic loss by using a sum of these two operation results;

S502, inputting a sample image of the second style into a second style transfer model containing the second residual generation network, to obtain a corresponding image of the first style; adding up an operation result obtained by inputting the image of the first style into the first residual generation network and an operation result obtained by inputting the sample image of the second style into the second residual generation network, and calculating a second cyclic loss by using a sum of these two operation results; and

S503, determining the cyclic loss function by using the first cyclic loss and the second cyclic loss.

The above-mentioned S501 and S502 are not limited in order of implementation, and may be implemented one after the other or synchronously.

In some embodiments, the parameters in the first residual generation network and the second residual generation network are adjusted to minimize the value of the cyclic loss function.

L_{GAN}: an adversarial loss, which is used to guide the generated residual to conform to characteristics of the target camera type. Two adversarial losses are shown in FIG. 4, namely, a first adversarial loss and a second adversarial loss.

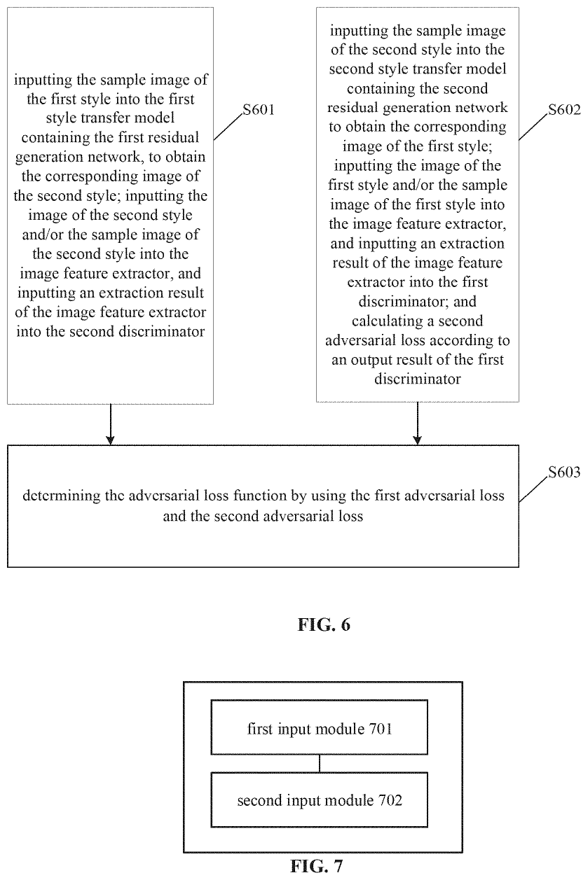

Referring to FIG. 4, in some embodiments, calculation of the adversarial loss function is shown in FIG. 6, including:

S601, inputting the sample image of the first style into the first style transfer model containing the first residual generation network, to obtain the corresponding image of the second style; inputting the image of the second style and/or the sample image of the second style into the image feature extractor, and inputting an extraction result of the image feature extractor into the second discriminator; and calculating a first adversarial loss according to an output result of the second discriminator;

S602, inputting the sample image of the second style into the second style transfer model containing the second residual generation network to obtain the corresponding image of the first style; inputting the image of the first style and/or the sample image of the first style into the image feature extractor, and inputting an extraction result of the image feature extractor into the first discriminator; and calculating a second adversarial loss according to an output result of the first discriminator; and

S603, determining the adversarial loss function by using the first adversarial loss and the second adversarial loss.

The above-mentioned S601 and S602 are not limited in order of implementation, and may be implemented one after the other or synchronously.

In some embodiments, an overall loss function is determined by using the adversarial loss function and the cyclic loss function;

parameters in the first discriminator and the second discriminator are adjusted when parameters in the first residual generation network and the second residual generation network are fixed, to maximize a value of the overall loss function; and

the parameters in the first residual generation network and the second residual generation network are adjusted when the parameters in the first discriminator and the second discriminator are fixed, to minimize the value of the overall loss function.

The optimization process of the loss function described above will be described in detail with reference to FIG. 4. In an embodiment of the application, the optimization of the loss function includes two aspects, namely the optimization of the cyclic loss function and the optimization of the adversarial loss function.

First aspect, optimization of the cyclic loss function:

As shown in FIG. 2, the original image a from the camera of type A is input to the residual generation network F, and the operation result is added to the original image a to obtain a_{B}. Similarly, the original image b from the camera of type B is input to the residual generation network G, and the operation result is added to the original image b to obtain b_{A}. The operation is specifically as follows:

a_{B}=a+λ₁R(a)

b_{A}=b+λ₂G(b)

Explicit segmentation of the image subjected to the style transfer into the original image and the residual can effectively preserve physiological structure information, and the influence of the residual on image structure authenticity can be further limited by limiting values of λ₁ and λ₂.

After that, the image a transferred to type B (i.e., a_{B}) is input to the residual generation network G, the operation result is added to F(a) to take an L2-norm, and an expected value is calculated for the result after taking the L2-norm to obtain the above-mentioned first cyclic loss. Symmetrically, the image b transferred to type A (i.e., b_{A}) is input to the residual generation network F, the operation result is added to G(b) to take an L2-norm, and an expected value is calculated for the result after taking the L2-norm to obtain the above-mentioned second cyclic loss. By adding the first cyclic loss and the second cyclic loss, the cyclic loss function may be obtained, specifically as follows:

L_{cyc}(F,G)= [symbol] [∥F(a)+G(a_{B})∥₂]+ [symbol] [∥G(b)+F(b_{A})∥₂]

Minimizing L_{cyc} during optimization can eliminate the difference between an image obtained by transferring a certain image of type A to type B-like through F and then further to type A through G and the original image as much as possible.

Second aspect, optimization of the adversarial loss function:

In order to make it easier for the discriminator to distinguish fundus images of different types (i.e., indirectly enhance the authenticity of camera type generation), the embodiment of the application chooses to add priori features related to the fundus image types, and constructs the image feature extractor f and the discriminator D (including D_{A} and D_{B}) separately. The feature extracted by the feature extractor at least includes the followings:

(1) mutual information among image channels;

(2) a colored histogram after image normalization; and

(3) a depth feature extracted from a fundus disease discrimination model, wherein the fundus disease discrimination model is the image classification model.

Optionally, the discriminators D_{A} and D_{B} are fully-connected shallow neural networks.

As shown in FIG. 2, the original image a from type A is input to the residual generation network F, and the operation result is added to the original image a to obtain a_{B}. And then a_{B} is input to the image feature extractor f, and the original image b from type B is input to the image feature extractor f. The extraction result of f is input into the discriminator D_{B}; and the first adversarial loss is calculated according to the output result of D_{B}. In this embodiment, the first adversarial loss is denoted as L_{GAN}(F, D_{B}). Symmetrically, the original image b from type B is input to the residual generation network G, and the operation result is added to the original image b to obtain b_{A}. And then b_{A} is input to the image feature extractor f, and the original image a from type A is input to the image feature extractor f. The extraction result of f is input into the discriminator D_{A}; and the second adversarial loss is calculated according to the output result of D_{A}. In this embodiment, the second adversarial loss is denoted as L_{GAN}(G, D_{A}).

The adversarial loss function includes the first adversarial loss and the second adversarial loss described above.

The overall loss function is determined by using the adversarial loss function and the cyclic loss function as follows:

$$L ( F , G , {D}_{A} , {D}_{B} ) = {L}_{GAN} ( F , {D}_{B} ) + {L}_{GAN} ( G , {D}_{A} ) + {λ}_{3} {L}_{cyc} ( F , G )$$

In some embodiments, the optimization of the overall loss function employs an adversarial mode of a minimax algorithm. D_{A} and D_{B} are adjusted when F and G are fixed, to maximize L(F, G, D_{A}, D_{B}), and F and G are adjusted when D_{A} and D_{B} are fixed, to minimize L(F, G, D_{A}, D_{B}).

That is, the object of the above optimization is as follows:

$${F}^{*} , {G}^{*} = arg {min}_{F , G} {max}_{{D}_{A} , {D}_{B}} L ( F , G , {D}_{A} , {D}_{B} )$$

And F* and G* obtained after optimization is a new first residual generation network and a new second residual generation network.

After a round of optimization, a batch of training samples may be selected and input again, the optimization including the above two aspects is repeated, and parameters are readjusted to cause the loss function to approach the optimal value. The training of the style transfer model proceeds until the value of the loss function reaches the desired range.

The image classification method and the style transfer model provided by the embodiments of the application may be applied to fundus disease screening. The embodiments of the application can realize the decoupling of the training process. That is, the model with well-trained laboratory data can also directly access the style transfer model provided by the embodiments of the application for use without requiring intervenes early in the training of the laboratory model. In addition, the embodiments of the application do not require professionally labeled data, and an unsupervised mode is adopted when the style transfer model provided by the embodiments of the application is trained with respect to the style transfer among various types of fundus cameras, that is, it's not necessary to know real disease diagnosis results of fundus images in various scenarios. In addition, the embodiments of the application can retain the main physiological structure information of the original image. With the style transfer model processed through the residuals, the original image structure information can be kept as properly as possible, and the consistency between the diagnosis results based on the image subjected to the style transfer and the original image is ensured. According to the embodiments of the application, the style transfer can be performed on a target fundus image, so that the target fundus image gets more similar to a sample of the training set in discrimination, and a deep learning discrimination model trained on a specific style data set can achieve higher accuracy on images of other styles.

An image classification apparatus is provided according to an embodiment of the application. FIG. 7 is a schematic diagram showing a structure of the image classification apparatus according to an embodiment of the application, the image classification apparatus including:

a first input module 701 configured for inputting an image of a first style into a style transfer model, to obtain an image of a second style corresponding to the image of the first style; and

a second input module 702 configured for inputting the image of the second style into an image classification model, to obtain a classification result of the image of the second style, wherein

the style transfer model is obtained through training on the basis of a sample image of the first style and a sample image of the second style; and the image classification model is obtained through training on the basis of the sample image of the second style.

In some embodiments, the image of the first style includes an image captured by a camera of a first type, and the image of the second style includes an image captured by a camera of a second type;

the image of the second style corresponding to the image of the first style includes an image having the same image content as the image of the first style and conforming to a shooting style of the camera of the second type.

In some embodiments, the image of the first style, the image of the second style, the sample image of the first style, and the sample image of the second style are all fundus images;

the style transfer model is obtained through training on the basis of the sample image of the first style without a disease label and the sample image of the second style with the disease label, in a manner of unsupervised adversarial training;

the image classification model is obtained through training on the basis of the sample image of the second style with the disease label, in a manner of supervised training.

In some embodiments, the style transfer model includes a residual generation network; the residual generation network includes a convolution layer and a pooling layer;

the image classification model includes a convolutional neural network.

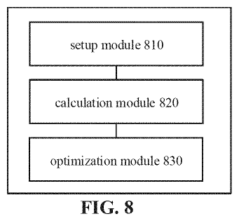

A style transfer model training apparatus is also provided according to an embodiment of the application. FIG. 8 is a schematic diagram showing a structure of a style transfer model training apparatus according to an embodiment of the application, the style transfer model training apparatus including:

a setup module 810 configured for setting a first residual generation network, a second residual generation network, an image feature extractor, a first discriminator and a second discriminator, wherein the first residual generation network is configured for generating a residual of a transfer from an image of a first style to an image of a second style; the second residual generation network is configured for generating a residual of a transfer from the image of the second style to the image of the first style; the image feature extractor is configured for extracting the feature information of the image of the first style or the image of the second style; the first discriminator is configured for determining whether an image is the image of the first style; and the second discriminator is configured for determining whether the image is the image of the second style;

a calculation module 820 configured for inputting training samples, and calculating a loss function by using output results of the first residual generation network, the second residual generation network, the first discriminator, and the second discriminator; and

an optimization module 830 configured for adjusting a parameter in at least one of the first residual generation network, the second residual generation network, the first discriminator, and the second discriminator to cause the value of the loss function to approach the optimal value.

In some embodiments, the loss function includes a cyclic loss function and a adversarial loss function.

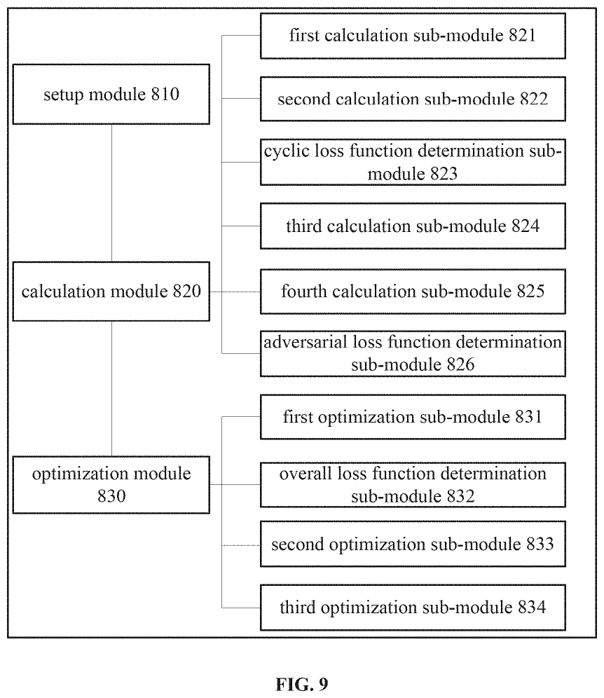

As shown in FIG. 9, in some embodiments, the calculation module 820 includes:

a first calculation sub-module 821 configured for inputting a sample image of the first style into a first style transfer model containing the first residual generation network, to obtain a corresponding image of the second style; adding up an operation result obtained by inputting the image of the second style into the second residual generation network and an operation result obtained by inputting the sample image of the first style into the first residual generation network, and calculating a first cyclic loss by using a sum of these two operation results;

a second calculation sub-module 822 configured for inputting a sample image of the second style into a second style transfer model containing the second residual generation network, to obtain a corresponding image of the first style; adding up an operation result obtained by inputting the image of the first style into the first residual generation network and an operation result obtained by inputting the sample image of the second style into the second residual generation network, and calculating a second cyclic loss by using a sum of these two operation results; and

a cyclic loss function determination sub-module 823 configured for determining the cyclic loss function by using the first cyclic loss and the second cyclic loss.

In some embodiments, the optimization module 830 includes:

a first optimization sub-module 831 configured for adjusting parameters in the first residual generation network and the second residual generation network, to minimize a value of the cyclic loss function.

In other embodiments, the calculation module 820 includes:

a third calculation sub-module 824 configured for inputting the sample image of the first style into the first style transfer model containing the first residual generation network to obtain the corresponding image of the second style; inputting the image of the second style and/or the sample image of the second style into the image feature extractor, and inputting an extraction result of the image feature extractor into the second discriminator; and calculating a first adversarial loss according to an output result of the second discriminator;

a fourth calculation sub-module 825 configured for inputting the sample image of the second style into the second style transfer model containing the second residual generation network, to obtain the corresponding image of the first style; inputting the image of the first style and/or the sample image of the first style into the image feature extractor, and inputting an extraction result of the image feature extractor into the first discriminator; and calculating a second adversarial loss according to an output result of the first discriminator; and

an adversarial loss function determination sub-module 826 configured for determining the adversarial loss function by using the first adversarial loss and the second adversarial loss.

In some embodiments, the optimization module 830 includes:

an overall loss function determination sub-module 832 configured for determining an overall loss function by using the adversarial loss function and the cyclic loss function;

a second optimization sub-module 833 configured for adjusting parameters in the first discriminator and the second discriminator when parameters in the first residual generation network and the second residual generation network are fixed, to maximize a value of the overall loss function; and

a third optimization sub-module 834 configured for adjusting the parameters in the first residual generation network and the second residual generation network when the parameters in the first discriminator and the second discriminator are fixed, to minimize the value of the overall loss function.

In some embodiments, the image feature extractor is configured for extracting at least one of:

mutual information among image channels;

a colored histogram after image normalization; and

a depth feature extracted from an image classification model.

The functions of modules in each apparatus of the embodiments of the application may refer to the corresponding description of the above methods, and will not be described in detail herein.

An electronic device and a readable storage medium are also provided according to the embodiments of the application.

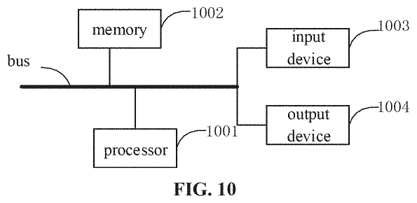

FIG. 10 is a block diagram of an electronic device for implementing embodiments of the application. The electronic device is intended to represent various forms of digital computers, such as laptop computers, desktop computers, workstations, personal digital assistants, servers, blade servers, mainframe computers, and other suitable computers. The electronic device may also represent various forms of mobile devices, such as personal digital processing, cellular telephones, smart phones, wearable devices, and other similar computing devices. The components shown herein, their connections and relationships, and their functions are by way of example only and are not intended to limit the implementations of the application described and/or claimed herein.

As shown in FIG. 10, the electronic device includes one or more processors 1001, a memory 1002, and interfaces for connecting components, including high-speed interfaces and low-speed interfaces. The various components are interconnected using different buses and may be installed on a common mainboard or otherwise as desired. The processor 1001 may process instructions for execution within the electronic device, including instructions stored in or on the memory 1002 to display graphical information of a GUI on an external input/output device 1004, such as a display device coupled to an interface. In other embodiments, multiple processors 1001 and/or multiple buses may be used with multiple memories 1002, if desired. Also, multiple connections with electronic devices are possible, each providing some of the necessary operations (e.g., as an array of servers, a group of blade servers, or a multiprocessor 1001 system). An example of one processor 1001 is shown in FIG. 10.

The memory 1002 is the non-transitory computer-readable storage medium provided herein. Wherein, the memory 1002 stores instructions executable by at least one processor 1001 to cause the at least one processor 1001 to perform the image classification method provided herein. The non-transitory computer-readable storage medium of the application stores computer instructions for causing a computer to perform the image classification method provided herein.

The memory 1002, as a non-transitory computer-readable storage medium, may be used to store non-transitory software programs, non-transitory computer-executable programs, and modules, such as program instructions/modules (e.g., the first input module 701 and the second input module 702 shown in FIG. 7, or the setup module 810, the calculation module 820 or the optimization module 830 shown in FIG. 8) corresponding to the image classification method or the style transfer model training method in the embodiments of the application. The processor 1001 executes various functional applications of the server and data processing, i.e., the image classification method in the method embodiments described above, by running non-transient software programs, instructions, and modules stored in the memory 1002.

The memory 1002 may include a program storage area and a data storage area, wherein the program storage area may store an operating system, an application program required for at least one function; the data storage area may store data created according to the use of the electronic device for image classification, etc. Additionally, the memory 1002 may include a high-speed random-access memory 1002, and may also include a non-transitory memory 1002, such as at least one disk memory 1002 piece, a flash memory piece, or other non-transitory solid-state memory 1002 pieces. In some embodiments, the memory 1002 may optionally include a memory 1002 remotely located with respect to the processor 1001, which may be connected to the electronic device for image classification via a network. Examples of such networks include, but are not limited to, the Internet, intranets, local area networks, mobile communication networks, and combinations thereof.

The electronic device for the image classification method may further include an input device 1003 and an output device 1004. The processor 1001, the memory 1002, the input device 1003, and the output device 1004 may be connected via a bus or otherwise, as exemplified in FIG. 10 via a bus connection.

The input device 1003 may receive input numeric or character information and generate key signal inputs related to user settings and functional controls of the electronic device for image classification, and examples of the input device 1003 include a touch screen, a keypad, a mouse, a track pad, a touch pad, a pointer, one or more mouse buttons, a track ball, a joystick, etc. The output device 1004 may include a display device, an auxiliary lighting device (e.g., an LED), a tactile feedback device (e.g., a vibration motor), etc. The display device may include, but is not limited to, a liquid crystal display (LCD), a light emitting diode (LED) display, and a plasma display. In some embodiments, the display device may be a touch screen.

Various embodiments of the systems and techniques described herein may be implemented in digital electronic circuits, integrated circuit systems, ASICs (application specific integrated circuits), computer hardware, firmware, software, and/or combinations thereof. These various embodiments may include implementations in one or more computer programs, which may be executed and/or interpreted on a programmable system including at least one programmable processor 1001, the programmable processor 1001 may be a specific or general purpose programmable processor 1001, may receive data and instructions from, and transmit data and instructions to, a memory system, at least one input device 1003, and at least one output device 1004.

These computing programs (also referred to as programs, software, software applications, or code) include machine instructions for the programmable processor 1001, and may be implemented using advanced procedural-oriented and/or object-oriented programming languages, and/or assembly/machine languages. As used herein, the terms “machine-readable medium” and “computer-readable medium” refer to any computer program product, device, and/or device (e.g., magnetic disks, optical disks, the memory 1002, programmable logic device (PLD)) for providing the programmable processor 1001 with machine instructions and/or data, including machine-readable media that receive machine instructions as machine-readable signals. The term “machine-readable signal” refers to any signal used to provide the programmable processor 1001 with machine instructions and/or data.

To provide interaction with a user, the systems and techniques described herein may be implemented on a computer having a display device (e.g., a CRT or LCD monitor) for displaying information to a user; and a keyboard and a pointer device (e.g., a mouse or a trackball) through which a user can provide input to the computer. Other types of devices may also be used to provide interaction with a user, for example, the feedback provided to the user may be any form of sensory feedback (e.g., visual feedback, auditory feedback, or tactile feedback); and input from the user may be received in any form, including acoustic input, voice input, or tactile input.

The systems and techniques described herein may be implemented in a computing system that includes a background component (e.g., as a data server), or a computing system that includes a middleware component (e.g., an application server), or a computing system that includes a front-end component (e.g., a user computer having a graphical user interface or a web browser through which a user may interact with embodiments of the systems and techniques described herein), or in a computing system that includes any combination of such background components, middleware components, or front-end components. The components of the system may be interconnected by any form or medium of digital data communication (e.g., a communication network). Examples of the communication network include Local Area Networks (LANs), Wide Area Networks (WANs), and the Internet.

The computer system may include a client and a server. The client and the server are typically remote from each other and typically interact through the communication network. The relationship between the client and the server is generated by computer programs running on respective computers and having a client-server relationship with each other. The server can be a cloud server, also called a cloud computing server or a cloud host, which is a host in a cloud computing service system, solving the defects of high difficulties in management and poor business expansibility in the conventional physical host and VPS service.

According to the technical solution of the embodiments of the application, the image of the first style is subjected to the style transfer implemented by the style transfer model, and the obtained image of the second style is input to the image classification model; the image classification model is obtained through training on the basis of the sample image of the second style. Since the style of the image processed by the image classification model in actual image classification is consistent with the style of the image employed in training, the reduced accuracy of the deep learning model due to the difference between the training set and the actual scenario is avoided, and the significant inferiority of the accuracy of the deep learning model in the actual scenario to that in the laboratory is prevented, hence the accuracy of image classification is improved.

It will be appreciated that the various forms of processes can me employed by reordering, adding, or removing the steps. For example, the steps recited in the application may be performed in parallel, sequentially or in a different order, so long as the desired results of the technical solution disclosed in the application can be achieved, and no limitation is made herein.

The above-described embodiments are not to be construed as limiting the scope of the application. It will be apparent to those skilled in the art that various modifications, combinations, sub-combinations, and substitutions are possible, depending on design requirements and other factors. Any modifications, equivalents, and improvements within the spirit and principles of this application are intended to be included within the scope of this application.

What is claimed is:

1. An image classification method, comprising:
inputting an image of a first style into a style transfer model, to obtain an image of a second style corresponding to the image of the first style; and
inputting the image of the second style into an image classification model, to obtain a classification result of the image of the second style, wherein
the style transfer model is obtained through training on the basis of a sample image of the first style and a sample image of the second style; and the image classification model is obtained through training on the basis of the sample image of the second style.

2. The method according to claim 1, wherein the image of the first style comprises an image captured by a camera of a first type, and the image of the second style comprises an image captured by a camera of a second type;
the image of the second style corresponding to the image of the first style comprises: an image having a same image content as the image of the first style and conforming to a shooting style of the camera of the second type.

3. The method according to claim 1, wherein the image of the first style, the image of the second style, the sample image of the first style, and the sample image of the second style are all fundus images;
the style transfer model is obtained through training on the basis of the sample image of the first style without a disease label and the sample image of the second style with the disease label, in a manner of unsupervised adversarial training;
the image classification model is obtained through training on the basis of the sample image of the second style with the disease label, in a manner of supervised training.

4. The method according to claim 1, wherein the style transfer model comprises a residual generation network; the residual generation network comprises a convolution layer and a pooling layer;
the image classification model comprises a convolutional neural network.

5. A style transfer model training method, comprising:
setting a first residual generation network, a second residual generation network, an image feature extractor, a first discriminator and a second discriminator, wherein the first residual generation network is configured for generating a residual of a transfer from an image of a first style to an image of a second style; the second residual generation network is configured for generating a residual of a transfer from the image of the second style to the image of the first style; the image feature extractor is configured for extracting feature information of the image of the first style or the image of the second style; the first discriminator is configured for determining whether an image is the image of the first style; and the second discriminator is configured for determining whether the image is the image of the second style;
inputting training samples, and calculating a loss function by using output results of the first residual generation network, the second residual generation network, the first discriminator, and the second discriminator; and
adjusting a parameter in at least one of the first residual generation network, the second residual generation network, the first discriminator and the second discriminator, to cause a value of the loss function to approach an optimal value.

6. The method according to claim 5, wherein the loss function comprises a cyclic loss function and an adversarial loss function.

7. The method according to claim 6, wherein calculating the cyclic loss function comprises:
inputting a sample image of the first style into a first style transfer model containing the first residual generation network, to obtain a corresponding image of the second style; adding up an operation result obtained by inputting the image of the second style into the second residual generation network and an operation result obtained by inputting the sample image of the first style into the first residual generation network, and calculating a first cyclic loss by using a sum of these two operation results;
inputting a sample image of the second style into a second style transfer model containing the second residual generation network, to obtain a corresponding image of the first style; adding up an operation result obtained by inputting the image of the first style into the first residual generation network and an operation result obtained by inputting the sample image of the second style into the second residual generation network, and calculating a second cyclic loss by using a sum of these two operation results; and
determining the cyclic loss function by using the first cyclic loss and the second cyclic loss.

8. The method according to claim 7, wherein the adjusting the parameter in the at least one of the first residual generation network, the second residual generation network, the first discriminator, and the second discriminator, to cause the value of the loss function to approach the optimal value, comprises:
adjusting parameters in the first residual generation network and the second residual generation network, to minimize a value of the cyclic loss function.

9. The method according to claim 7, wherein calculating the adversarial loss function comprises:
inputting the sample image of the first style into the first style transfer model containing the first residual generation network, to obtain the corresponding image of the second style; inputting the image of the second style and/or the sample image of the second style into the image feature extractor, and inputting an extraction result of the image feature extractor into the second discriminator; and calculating a first adversarial loss according to an output result of the second discriminator;
inputting the sample image of the second style into the second style transfer model containing the second residual generation network, to obtain the corresponding image of the first style; inputting the image of the first style and/or the sample image of the first style into the image feature extractor, and inputting an extraction result of the image feature extractor into the first discriminator; and calculating a second adversarial loss according to an output result of the first discriminator; and
determining the adversarial loss function by using the first adversarial loss and the second adversarial loss.

10. The method according to claim 9, wherein the adjusting the parameter in the at least one of the first residual generation network, the second residual generation network, the first discriminator, and the second discriminator, to cause the value of the loss function to approach the optimal value, comprises:
determining an overall loss function by using the adversarial loss function and the cyclic loss function;
adjusting parameters in the first discriminator and the second discriminator when parameters in the first residual generation network and the second residual generation network are fixed, to maximize a value of the overall loss function; and
adjusting the parameters in the first residual generation network and the second residual generation network when the parameters in the first discriminator and the second discriminator are fixed, to minimize the value of the overall loss function.

11. The method according to claim 5, wherein the image feature extractor is configured for extracting at least one of:
mutual information among image channels;
a colored histogram after image normalization; and
a depth feature extracted from an image classification model.

12. An image classification apparatus, comprising:
at least one processor; and
a memory communicatively connected with the at least one processor, wherein
the memory stores instructions executable by the at least one processor, and the instructions, when executed by the at least one processor, enable the at least one processor to perform operations comprising:
inputting an image of a first style into a style transfer model, to obtain an image of a second style corresponding to the image of the first style; and
inputting the image of the second style into an image classification model, to obtain a classification result of the image of the second style, wherein
the style transfer model is obtained through training on the basis of a sample image of the first style and a sample image of the second style; and the image classification model is obtained through training on the basis of the sample image of the second style.

13. The apparatus according to claim 12, wherein the image of the first style comprises an image captured by a camera of a first type, and the image of the second style comprises an image captured by a camera of a second type;
the image of the second style corresponding to the image of the first style comprises: an image having a same image content as the image of the first style and conforming to a shooting style of the camera of the second type.

14. The apparatus according to claim 12, wherein the image of the first style, the image of the second style, the sample image of the first style, and the sample image of the second style are all fundus images;
the style transfer model is obtained through training on the basis of the sample image of the first style without a disease label and the sample image of the second style with the disease label, in a manner of unsupervised adversarial training;
the image classification model is obtained through training on the basis of the sample image of the second style with the disease label, in a manner of supervised training.

15. The apparatus according to claim 12, wherein the style transfer model comprises a residual generation network; the residual generation network comprises a convolution layer and a pooling layer;
the image classification model comprises a convolutional neural network.

16. A style transfer model training apparatus, comprising:
at least one processor; and
a memory communicatively connected with the at least one processor, wherein
the memory stores instructions executable by the at least one processor, and the instructions, when executed by the at least one processor, enable the at least one processor to perform operations comprising:
setting a first residual generation network, a second residual generation network, an image feature extractor, a first discriminator and a second discriminator, wherein the first residual generation network is configured for generating a residual of a transfer from an image of a first style to an image of a second style; the second residual generation network is configured for generating a residual of a transfer from the image of the second style to the image of the first style; the image feature extractor is configured for extracting feature information of the image of the first style or the image of the second style; the first discriminator is configured for determining whether an image is the image of the first style; and the second discriminator is configured for determining whether the image is the image of the second style;
inputting training samples, and calculating a loss function by using output results of the first residual generation network, the second residual generation network, the first discriminator, and the second discriminator; and
adjusting a parameter in at least one of the first residual generation network, the second residual generation network, the first discriminator, and the second discriminator, to cause a value of the loss function to approach an optimal value.

17. The apparatus according to claim 16, wherein the loss function comprises a cyclic loss function and an adversarial loss function.

18. The apparatus according to claim 17, wherein calculating the cyclic loss function comprises:
inputting a sample image of the first style into a first style transfer model containing the first residual generation network, to obtain a corresponding image of the second style;
adding up an operation result obtained by inputting the image of the second style into the second residual generation network and an operation result obtained by inputting the sample image of the first style into the first residual generation network, and calculating a first cyclic loss by using a sum of these two operation results;
inputting a sample image of the second style into a second style transfer model containing the second residual generation network, to obtain a corresponding image of the first style; adding up an operation result obtained by inputting the image of the first style into the first residual generation network and an operation result obtained by inputting the sample image of the second style into the second residual generation network, and calculating a second cyclic loss by using a sum of these two operation results; and
determining the cyclic loss function by using the first cyclic loss and the second cyclic loss.

19. The apparatus according to claim 18, wherein the adjusting the parameter in the at least one of the first residual generation network, the second residual generation network, the first discriminator, and the second discriminator, to cause the value of the loss function to approach the optimal value, comprises:
adjusting parameters in the first residual generation network and the second residual generation network, to minimize a value of the cyclic loss function.

20. The apparatus according to claim 18, wherein calculating the adversarial loss function comprises:
inputting the sample image of the first style into the first style transfer model containing the first residual generation network, to obtain the corresponding image of the second style; inputting the image of the second style and/or the sample image of the second style into the image feature extractor, and inputting an extraction result of the image feature extractor into the second discriminator; and calculating a first adversarial loss according to an output result of the second discriminator;
inputting the sample image of the second style into the second style transfer model containing the second residual generation network, to obtain the corresponding image of the first style; inputting the image of the first style and/or the sample image of the first style into the image feature extractor, and inputting an extraction result of the image feature extractor into the first discriminator; and calculating a second adversarial loss according to an output result of the first discriminator; and
determining the adversarial loss function by using the first adversarial loss and the second adversarial loss.

21. The apparatus according to claim 20, wherein the adjusting the parameter in the at least one of the first residual generation network, the second residual generation network, the first discriminator, and the second discriminator, to cause the value of the loss function to approach the optimal value, comprises:
determining an overall loss function by using the adversarial loss function and the cyclic loss function;
adjusting parameters in the first discriminator and the second discriminator when parameters in the first residual generation network and the second residual generation network are fixed, to maximize a value of the overall loss function; and
adjusting the parameters in the first residual generation network and the second residual generation network when the parameters in the first discriminator and the second discriminator are fixed, to minimize the value of the overall loss function.

22. The apparatus according to claim 16, wherein the image feature extractor is configured for extracting at least one of:
mutual information among image channels;
a colored histogram after image normalization; and
a depth feature extracted from an image classification model.

23. A non-transitory computer-readable storage medium storing computer instructions, wherein the computer instructions cause a computer to perform operations comprising:
inputting an image of a first style into a style transfer model, to obtain an image of a second style corresponding to the image of the first style; and
inputting the image of the second style into an image classification model, to obtain a classification result of the image of the second style, wherein
the style transfer model is obtained through training on the basis of a sample image of the first style and a sample image of the second style; and the image classification model is obtained through training on the basis of the sample image of the second style.
